(19) United States
(12) Patent Application Publication
(10) Pub. No.: US 2009/0105669 A1
(43) Pub. Date: Apr. 23, 2009

(54) METHOD AND APPARATUS FOR TREATING SUBJECTS SUFFERING FROM CONTRACTURE

(76) Inventors: Patricia Rose Smigielski, Arlington, TX (US); Dorothy K. Monfre, Gilbert, AZ (US); Stephen L. Monfre, Gilbert, AZ (US)

Correspondence Address: GLENN PATENT GROUP, 3475 EDISON WAY, SUITE L, MENLO PARK, CA, 94025, US

(21) Appl. No.: 11/874,851

(22) Filed: Oct. 18, 2007

(51) Int. Cl. A61M 35/00

(52) U.S. Cl. 604/290; 604/289

(57) ABSTRACT

A method and apparatus for cleaning a subject suffering from contracture provides a threaded and flexible cleaning tool for insertion between muscles joints or body parts suffering from contracture requiring no prying open of joints or minimally prying open of joints in order to facilitate cleaning of body parts otherwise obstructed by the contracture.

### BACKGROUND OF THE INVENTION

1. Technical Field

The invention relates to a medical condition. More particularly, the invention relates to a method and apparatus for treating subjects who suffer from contracture.

2. Description of the Related Art

There exists a medical disorder of contracture, which results from a number of sources. For example, after a stroke or other neurological disorder, tightness of the hand muscles of an individual often lead to debilitating contracture of the fingers of the hand, resulting in a fist-like deformity. While this condition can be treated by the use of splinting, the treatment is painful and the patient is often unwilling to go through painful procedures, such as stretching and range of motion exercises, required to treat the contracture. The contracture or tightness, if not treated often becomes worse and can lead to the finger nails puncturing the flesh. Further, infection of palm and spaces between the finger and thumb often results due to difficulties in cleaning, medicating, drying or lubricating the tightly fisted hand.

Health practitioners, such as occupational therapists, physical therapists, and nursing staff often try to wedge open the hand of a subject suffering from contracture to insert a folded washcloth or roll of gauze bandage. Other common means of keeping the hand open to prevent further contracture include the use of conical tubes and palm splints. Cone shaped tubes are generally made of rigid plastic and may be covered with some sort of thin material to make them more comfortable. Often lengths from four to five inches and widths tapering from three quarters of an inch to one and a half inches in diameter are used. A palm splint fits into the palm, and rests between the fingers and the palm surface, acting as a barrier to prevent the fingers from digging into the palm of the skin. In extreme cases, surgery where tendons are severed is a last, painful, and often ineffective resort.

It is a particular problem with all of the known devices for treating contracture that their use requires the fingers to be pried open before inserting the device into the tightly clenched fist. This opening of the fingers is typically extremely painful and in some cases can cause dislocation of a joint, even when performed by a therapist, medical practitioner, or family caregiver. The required force exerted to pry open the fingers or to open the hand to insert a splint and/or the aforementioned cone devices is often considerable as the force required must open the hand or fingers sufficiently to insert a cone through the widest opening of the clenched fist toward the narrowest opening of the fingers. To insert even a gauze roll is typically painful and traumatic for the patient because of the necessity of prying the fingers. The prying of the fingers apart and prying to open the palm of the hand is also necessary for the simple act of cleaning of the palm and fingers with a hand towel.

What is needed is a method and apparatus that facilitates cleaning of the palm and fingers of a subject suffering from contracture that does not necessitate prying open the palm of the hand and fingers of subjects suffering from contracture.

### SUMMARY OF THE INVENTION

The invention provides a method and apparatus for cleaning, e.g. the hand of, a subject suffering from contracture. More particularly, the invention provides a threaded and flexible cleaning tool for insertion between muscles joints or body parts suffering from contracture requiring no prying open of joints or minimally prying open of joints in order to facilitate cleaning of body parts otherwise obstructed by the contracture.

### BRIEF DESCRIPTION OF THE DRAWINGS

FIG. 1 provides a block diagram of an apparatus used to facilitate cleaning of body parts obstructed by contracture according to the invention.

### DETAILED DESCRIPTION OF THE INVENTION

Herein, a contracture is an abnormal often permanent shortening of a muscle or scar tissue resulting in distortion or deformity of a joint or appendage of the body. A contracture results in a permanent or long term shortening of a muscle, tendon, or scar tissue producing a deformity or distortion. A contracture is an abnormal and usually permanent contraction of a muscle.

In one embodiment of the invention, a cleaning tool is attached or integrated into a flexible longitudinal translation means inserted into the narrow openings left by the contracture between the digits of the hand such as the fingers and/or thumb, and palm. The cleaning tool is preferably includes a member set of flexible or deformable cleaning elements, such as tabs, extending radially off of the longitudinal translation means. One end of the longitudinal translation means is threaded between two body parts into an area obstructed by contracture. The cleaning tool is then pulled through the obstructed area contacting the skin surface of the obstructed areas with the flexible cleaning elements thereby cleaning the hard to reach skin surface areas obstructed by the contracture. Optionally, both ends of the longitudinal translation means have means for holding allowing the cleaning tool to be translated back and forth through the obstructed region in a flossing manner allowing multiple cleaning passes on the obstructed area.

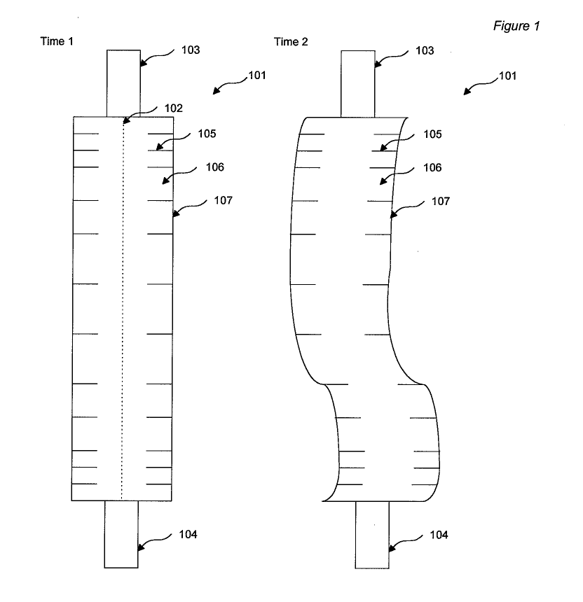

Referring now to FIG. 1, a block diagram of an exemplary version of the cleaning apparatus is provided. The cleaning tool 101 has a longitudinal axis 102 having a first and second end. Preferably, at least one of the first and second ends has a narrow cross-section tab for facilitation of translating the cleaning tool between obstructed body parts. In FIG. 1, the first end 103 and second end 104 of the cleaning tool both have tabs and/or a grasping element to facilitate translation. Attached or integrated into the longitudinal translation means are radially extending cleaning surfaces 107. Both the longitudinal axis and the radially extending cleaning elements are either deformable or flexible to facilitate pulling the cleaning tool through tight and/or curved passages formed by the contracture, such as between fingers or along the palm. The cleaning tool is illustrated at time 1 in an elongated shape and at time 2 in a curved shape to illustrate the flexibility of the tool.

The elongated longitudinal axis is preferably a thin material that is both readily grasped by the person cleaning and is narrow and rigid enough for ease of threading between body parts such as fingers, toes, or a fist along the palm. Preferably, the longitudinal axis of the cleaning tool is uniform in cross-section and has a diameter of about 1/116^{th}, 2/16^{th}, 3/16^{th}, 4/16^{th}, 5/16^{th}, 6/16^{th}, 7/16^{th}, 8/16^{th}, 9/16^{th}, 10/16^{th}, 11/16^{th}, or 12/16^{th} of and inch in its cross-sectionally compressed form. Integrated or radiating from the longitudinal axis are cleaning elements. Preferably the cleaning elements have frayed or fringed edges to maximize surface area contact and thus cleaning ability of the skin. In one example, the cleaning elements are a sponge-like material that deforms and expands during translation according to the applied pressure and gaps of the body parts, such as fingers, created by the contracture. In this manner, the cleaning tool passes through and cleans narrow openings created by contracture while at the same time expanding and cleaning larger openings within the volume created by the contracture. In a second example, the cleaning elements are a plurality of radially extending tabs 106 separated by cuts 105. The tabs are either orthogonal to the longitudinal axis or at an angle to the longitudinal axis. The tabs are either uniform in size of are of varying size. An example of a tab is a soft terry-type cloth stitched together in multiple layers. The cloth is then snipped at intervals yielding a fringed edge. The intervals are either uniformly or non-uniformly spaced throughout the longitudinal axis or about some midpoint. Preferably, the cleaning elements extend in three-dimensions from the longitudinal axis; however, two-dimensional extensions of tabs is also useful for cleaning and/or treating smaller cavities left by contracture.

In another embodiment of the invention, the radius or extension away from the center of the cleaning tool in non-uniform along the longitudinal axis. Herein, radius is used generically as the extension from the center longitudinal axis, though the cross sectional area can be of an shape and is not limited to a circular cross section. For example, the tool is tapered from a small radius, such as about 1/16^{th} inch, at the first end to a larger radius, such as about 12/16^{th} inch, at the second end. This allows insertion and translation until resistance thereby optimizing the cleaning cross-section of the tool in the opening formed by the contracture. In a second example, the tool has cross-sectional areas that are a step function along the longitudinal axis. For instance, one longitudinal part of the tool has a cross sectional area that is different from the cross-sectional area of a second longitudinal part of the tool. This facilitates cleaning by a pulsing force applied to the skin as the cleaning tool is translated along the skin.

In yet another embodiment of the invention, the elements are coated, partially encapsulated, and/or impregnated with an agent, such as soap, lotion, or medication to assist in the cleaning, medicating, localized drug delivery, lubricating, and care of the obstructed body part, such as hands and fingers. The coating agent could be a pure component, a compound, a solution, or a mixture. Optionally, the cloth can be pure to allow for the drying of treated hands and fingers.

Furthermore, the obstructed body part could also consist of an arm, upper arm, torso, leg or digit of the foot such as a toe. An obstruction could be circumferentially or partial enclosure that is either inward or outward. Optionally, the obstruction could contain blistering of the skin, sores on the skin, and/or fungal growth on the skin.

In view of the different possible embodiments to which the principle of this invention may be applied, it should be recognized that the preferred embodiment described herein with respect to the drawings is meant to the illustrative only and should not be taken as limiting the scope of the invention. One skilled in the art will readily appreciate that other applications may be substituted for those set forth herein without departing from the spirit and scope of the present invention. Accordingly, the invention should only be limited by the Claims included below.

1. A method for treating a human subject suffering from contracture, comprising the steps of:
providing a flexible, longitudinal member, wherein said flexible longitudinal member further comprises a plurality of tabs extending radially from said longitudinal member;
threading said member through an opening defined by at least one appendage of the subject, said appendage having an external surface comprised, at least in part, of skin; and
flossing at least a portion of said external surface with said tabs of said longitudinal member;
wherein said flossing facilitates treatment of the human subject.

2. The method of claim 1, wherein said step of providing a member further comprises the step of providing at least one of:
a cleaning element;
a drying element;
a medicating element; and
a lubricating element.

3. The method of claim 1, wherein said appendage comprises one or more of:
a digit of a hand;
a finger;
a thumb;
a palm;
an arm;
a leg;
a digit of a toe;
a toe;
an upper arm; and
a torso.

4. The method of claim 3, wherein said at least one appendage circumferentially encloses said opening.

5. The method of claim 3, wherein said appendage partially encloses said opening.

6. The method of claim 1, wherein said external surface comprises an outer layer of skin.

7. The method of claim 1, wherein said step of flossing comprises the step of relieving symptoms related to said contracture, wherein said symptoms comprise any of:
blistering of the skin;
sores on the skin; and
fungal growth on the skin.

8. An apparatus for treating a human subject suffering from contracture, comprising:
a flexible longitudinal member, wherein said flexible longitudinal member further comprises a plurality of tabs extending outward from said longitudinal member,
wherein said plurality of tabs are at least partially treated with a coating element comprising at least one of:
a cleaning element;
a drying element;
a medicating element; and
a lubricating element.

9. The apparatus of claim 8, wherein said coating element at least partially contacts said longitudinal member.

10. The apparatus of claim 8, wherein said coating element comprises at least one of:
a compound;
a solution;
a mixture; and
a pure component.

11. The apparatus of claim 10, wherein said coating element contacts with said plurality of tabs through any of:
impregnation;
coating; and
partial encapsulation.

12. The apparatus of claim 10, wherein at least one of said plurality of tabs comprises a frayed edge that increases surface area of said plurality of tabs.

13. The apparatus of claim 10, wherein said plurality of tabs are either uniformly or non-uniformly spaced along said longitudinal member.

14. The apparatus of claim 10, wherein a first tab of said plurality of tabs comprises a first surface area, wherein a second tab of said plurality of tabs comprises a second surface area, wherein said second surface area is at least twenty percent larger than said first tab.

15. The apparatus of claim 10, wherein said longitudinal member comprises a first end, a second end, and a midpoint.

16. The apparatus of claim 15, wherein a first tab about said midpoint is at least thirty percent larger than a second tab proximate said first end.

17. The apparatus of claim 15, further comprising a grasping element connected to said first end.

18. An apparatus for treating a human subject suffering from contracture, comprising:
a flexible longitudinal member at least partially treated with a coating element comprising at least one of:
a cleaning element;
a medicating element; and
a lubricating element;
wherein flossing the human subject with said member aids in treatment of contracture.

19. The apparatus of claim 18, wherein said flexible longitudinal member further comprises a plurality of tabs extending outward from said longitudinal member.

20. The apparatus of claim 18, wherein at least one of said longitudinal member and said plurality of tabs comprises material that is deformable under pressures applied during said flossing.

21. An apparatus, comprising:
a flexible longitudinal member adapted for insertion through an opening defined by an appendage and for any of axial and circumferential movement therein, wherein said flexible longitudinal member further comprises a plurality of tabs extending radially outward from an axis of said longitudinal member, said tabs adapted for contact with an appendage surface within said opening;
wherein said plurality of tabs comprise a flossing element;
wherein said flossing element comprises a material that is deformable under pressure.

22. The apparatus of claim 21, further member further comprising at least one of:
a cleaning element;
a medicating element; and
a lubricating element.
